(19) United States
(12) Patent Application Publication
(10) Pub. No.: US 2021/0391046 A1
(43) Pub. Date: Dec. 16, 2021

(54) A SYSTEM AND METHOD FOR MEDICAL VISIT DOCUMENTATION AUTOMATION AND BILLING CODE SUGGESTION IN CONTROLLED ENVIRONMENTS

(71) Applicant: KONINKLIJKE PHILIPS N.V., ENDHOVEN (NL)

(72) Inventors: Mladen MILOSEVIC, Stoneham, MA (US); Daniel Jason SCHULMAN, Jamaica Plain, MA (US); Christine Menking SWISHER, San Diego, CA (US)

(21) Appl. No.: 17/286,257

(22) PCT Filed: Oct. 15, 2019
(86) PCT No.: PCT/EP2019/077881

§ 371 Date: Apr. 16, 2021

Related U.S. Application Data
(60) Provisional application No. 62/746,242, filed on Oct. 16, 2018.

(51) Int. Cl. G16H 15/00; G16H 10/60; G06F 40/58

(52) U.S. Cl. CPC G16H 15/00; G06F 40/58; G16H 10/60

(57) ABSTRACT

Various embodiments relate to a method and system for automatically generating a medical document during a medical visit in a controlled environment, the method including the steps of monitoring, by a network monitoring module, a network to capture use of medical equipment connected to the network, detecting, by an atomic action video recognition module, predefined atomic actions in the controlled environment, extracting, by a patient-medical provider conversation recognition module, clinical information from a conversation between a patient and a medical provider, matching, by a visit graph generation module, the use of medical equipment and the predefined atomic actions to an atomic actions and CPT codes database of known uses of medical equipment and predefined atomic actions, generating, by the visit graph generation module, an event graph based on the use of medical equipment, the predefined atomic actions and the extracted clinical information and translating, by a medical document generator, the event graph into a medical document.

### TECHNICAL FIELD

This disclosure relates generally to medical documentation, and more specifically, but not exclusively, to automation of medical documentation in a controlled environment.

### BACKGROUND

Medical personnel are required to maintain documentation for a patient medical visit. For patients, accurate medical documentation improves the quality of the care and provides continuity of care because the medical documentation creates a medical history and also a means of communication between health care providers and insurance companies about current health status, treatment and delivery of care. For medical providers, accurate medical documentation of their findings and course of actions provides a record that serves as a justification for procedural charges they submit to payers.

However, maintaining well written visit documentation may be time consuming and may require substantial attention from the provider, which may decrease the medical provider's efficiency in providing care and may be disruptive to the workflow. For example, if a medical provider documents during a patient visit, this may lengthen the visit and disrupt patient-provider interactions, both of which may decrease patient satisfaction. Providers may document after the visit which may lead to an error or omission.

### SUMMARY

A brief summary of various embodiments is presented below. Embodiments address a system and method for medical visit documentation and automation and billing code suggestion in environment.

A brief summary of various example embodiments is presented. Some simplifications and omissions may be made in the following summary, which is intended to highlight and introduce some aspects of the various example embodiments, but not to limit the scope of the invention.

Detailed descriptions of example embodiments adequate to allow those of ordinary skill in the art to make and use the inventive concepts will follow in later sections.

Various embodiments described herein relate to a method for automatically generating a medical document during a medical visit in a controlled environment, the method including the steps of monitoring, by a network monitoring module, a network to capture use of medical equipment connected to the network, detecting, by an atomic action video recognition module, predefined atomic actions in the controlled environment, extracting, by a patient-medical provider conversation recognition module, clinical information from a conversation between a patient and a medical provider, matching, by a visit graph generation module, the use of medical equipment and the predefined atomic actions to an atomic actions and CPT codes database of known uses of medical equipment and predefined atomic actions, generating, by the visit graph generation module, an event graph based on the use of medical equipment, the predefined atomic actions and the extracted clinical information and translating, by a medical document generator, the event graph into a medical document.

In an embodiment of the present disclosure, the method for automatically generating a medical document during a medical visit in a controlled environment, the method further including the steps of transmitting, by a communication interface, the medical document to the medical provider for review.

In an embodiment of the present disclosure, the medical document generator improves the translation of the event graph into the medical document by capturing the changes made by the medical provider.

In an embodiment of the present disclosure, the network monitoring module monitors the use of medical equipment by monitoring transactions in an electronic medical record (“EMR”) to extract the use of medical equipment.

In an embodiment of the present disclosure, the patient-medical provider conversation recognition module extracts clinical information from the conversation by using an algorithm to differentiate the patient and the medical provider, extracting features from the conversation, decoding phonemes from the conversation to raw text, using natural language processing (“NLP”) to convert the raw text to processed text and mapping the processed text into a plurality of concepts using clinical ontologies.

In an embodiment of the present disclosure, the event graph includes connecting the atomic actions detected by the atomic actions video recognition module to other atomic actions using temporal ordering.

In an embodiment of the present disclosure, the visit graph generation module uses a template event graph to generate the event graph.

In an embodiment of the present disclosure, the medical document generator categorized the concepts from the event graph into categories based on a similarity score for each of the plurality of concepts using recurrent neural networks.

In an embodiment of the present disclosure, the medical document generator uses template based slot filling to generate the medical document from the categories.

In an embodiment of the present disclosure, the medical document generator proposes a current procedural terminology code for the medical visit based on the medical document.

Various embodiments described herein relate to a system for automatically generating a medical document during a medical visit in a controlled environment, the system including a network monitoring module configured to monitor a network to capture use of medical equipment connected to the network, an atomic action video recognition module configured to detect predefined atomic actions in the controlled environment, a patient-medical provider conversation recognition module configured to extract clinical information from a conversation between a patient and a medical provider, a visit graph generation module configured to match the use of medical equipment and the predefined atomic actions to an atomic actions and CPT codes database of known uses of medical equipment and predefined atomic actions, the visit graph generation module configured to generate an event graph based on the use of medical equipment, the predefined atomic actions and the extracted clinical information and a medical document generator configured to translate the event graph into a medical document.

In an embodiment of the present disclosure, the system for automatically generating a medical document during a medical visit in a controlled environment, the system further including a communication interface configured to transmit the medical document to the medical provider for review.

In an embodiment of the present disclosure, the medical document generator improves the translation of the event graph into the medical document by capturing the changes made by the medical provider.

In an embodiment of the present disclosure, the network monitoring module monitors the use of medical equipment by monitoring transactions in an electronic medical record (“EMR”) to extract the use of medical equipment.

In an embodiment of the present disclosure, the patient-medical provider conversation recognition module extracts clinical information from the conversation by using an algorithm to differentiate the patient and the medical provider, extracting features from the conversation, decoding phonemes from the conversation to raw text, using natural language processing (“NLP”) to convert the raw text to processed text and mapping the processed text into a plurality of concepts using clinical ontologies.

In an embodiment of the present disclosure, the event graph includes connecting the atomic actions detected by the atomic actions video recognition module to other atomic actions using temporal ordering.

In an embodiment of the present disclosure, the visit graph generation module uses a template event graph to generate the event graph.

In an embodiment of the present disclosure, the medical document generator categorized the concepts from the event graph into categories based on a similarity score for each of the plurality of concepts using recurrent neural networks.

In an embodiment of the present disclosure, the medical document generator uses template based slot filling to generate the medical document from the categories.

In an embodiment of the present disclosure, the medical document generator proposes a current procedural terminology code for the medical visit based on the medical document.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates a block diagram of the system for medical visit documentation automation and billing code suggestion in a controlled environment of the current embodiment;

FIG. 2 illustrates a schema for medical data information recorded from multiple data streams of patient-medical provider interaction to relevant information and events during visit of the current embodiment;

FIG. 3 illustrates a schema of a history and physical (“H&P”) generator from Visit Graph of the current embodiment; and

FIG. 4 illustrates a block diagram of a real-time data processing system of the current embodiment.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments. Descriptors such as “first,” “second,” “third,” etc., are not meant to limit the order of elements discussed, are used to distinguish one element from the next, and are generally interchangeable.

Documenting a medical visit may use either templates which the medical provider may amend and correct to create an accurate record of the medical visit or may use voice dictation either during or after the medical visit to create an accurate record of the medical visit.

When using a template, the medical provider selects a specific template and then amends sections which are specific for the current patient. Templates may introduce errors in clinical notes since a medical provider may not correctly change a section of the defaulted text of the template.

Furthermore, including templates for a large spectrum of clinical notes and circumstances also may introduces difficulties. Assuming templates are available for widely known circumstances, entering clinical notes for infrequent medical procedures, which are not covered by a template, may be comparatively time-consuming, which may cause a medical provider who is under time pressure to see more patients to be careless with entering these clinical notes which may raise the risk of an error or omission in the clinical notes. Further, with a large group of clinical notes templates, it may become difficult for a medical provider to be aware of all available clinical notes templates and may be more time consuming to find and use an appropriate clinical note template.

When using voice dictation, the medical provider may dictate about the medical visit and then voice recognition software may transcribe the recording into text. However, depending on the speaking and typing speeds, using voice dictation may or may not be more efficient (as compared to the medical provider typing clinical notes), especially if a medical provider must then spend time and effort checking for and correcting dictation errors by the software.

Using either clinical notes templates or voice dictation, are not proactive solutions and may require substantial effort and attention from medical providers.

The current embodiments address the need for a proactive solution which requires minimal effort from a medical provider. The current embodiments improve current documentation practices of clinical records in controlled environments (e.g., retail clinics) by automatically generating a draft of a medical visit case note, tailored for the current patient, at the time of visit. By generating the draft, the medical provider can then make modifications, if necessary, before saving the draft as the clinical notes.

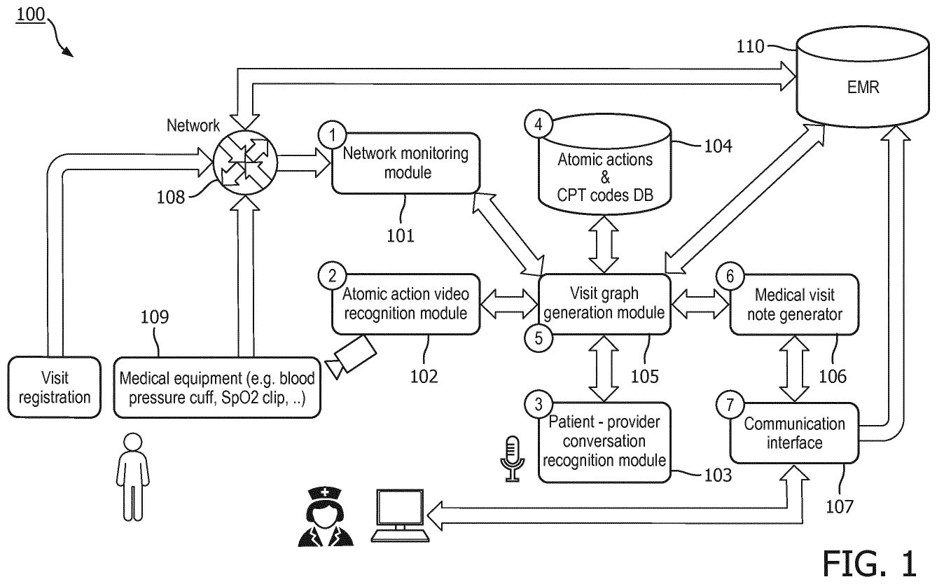

FIG. 1 illustrates a block diagram of the system 100 for medical visit documentation automation and billing code suggestion in a controlled environment of the current embodiment.

FIG. 1 of the system 100 includes a network monitoring module 101, an atomic action video recognition module 102, a provider conversation recognition module 103, an atomic actions and current procedural terminology (“CPT”) codes modules 104, a visit graph generation module 105, a medical document generator 106 and a communication interface 107.

The network monitoring module 101 monitors the network 108 to capture any use of the connected medical equipment 109 (i.e., blood pressure monitors, etc.).

The atomic action video recognition module 102 detects predefined “atomic actions” such as throat examination, from a real-time video of the medical provider's interaction with a patient in a constrained environment.

The patent-provider conversation recognition module 103 extracts relevant information from patient and medical provider conversation.

The atomic actions and CPT codes database 104 contains all possible atomic actions and CPT codes for a specific controlled environment.

The visit graph generation module 105 matches detected medical equipment 109 activity and atomic action detected from the video with all possible atomic actions from the atomic actions and CPT codes database 104 and generates an event graph for each visit.

The medical document generator 106 translates an event graph generated from the visit graph generation module 105 to a draft of medical note for a visit.

The communication interface 107 communicates a draft of the medical note to the healthcare medical provider and also captures changes the medical provider makes in order to allow for secondary uses such as improving generation in the future.

The current embodiment requires a controlled clinical environment, which is a set of integrated and networked clinical devices in a known configuration, which may be found in a retail clinic or other similar setup. The current embodiment uses a controlled clinical environment which allows for being aware of the capabilities of the controlled clinical environment (for example, being aware that a retail clinic cannot perform an orthopedic surgery), being aware of the patient's and medical provider's position within the environment and that statuses can be assessed continuously (e.g., there may be only one patient and one medical provider present in the clinical space).

The network monitoring module 101 monitors the use of medical equipment 109 during the medical visit which is performed by monitoring the network 108 over which the medical equipment 109 is communicating with the electronic medical records (“EMR”) 110. The network monitoring module 101 may transmit the detected events to the visit graph generation module 105.

For every detected event by the network monitoring module 101, the network monitoring module 101 may extract a type and model of the medical equipment, equipment configuration details if applicable, and measured value, if applicable (e.g., SpO2 clip or blood pressure cuff).

In an alternative embodiment, the network monitoring module 101 may monitor by monitoring the transactions in the EMR 110 and extract the same information as it would from the network 108.

The atomic action video recognition module 102 captures a medical provider's interaction with the patient that cannot be captured by the network monitoring module. The atomic action video recognition module 102 may use computer vision to detect predefined “atomic actions” of the medical provider, such as a visual inspection of torso by the medical provider, or a throat examination, from a real-time video of the medical provider's interaction with a patient in a constrained environment.

The patient-medical provider conversation recognition module 103 records information during the medical provider-patient interactions or the medical provider dictates directly to the medical visit note generator 106.

The communication interface module 107 communicates the draft of the medical note to a medical provider. The communication module 107 also captures changes the medical provider makes in order to improve generation of the draft of the medical note in the future, which allows the system 100 to improve by considering changes medical providers make to the generated draft medical note.

The improvement would not be limited to generation of draft of the medical note from an event graph, but also to generation of the event graph.

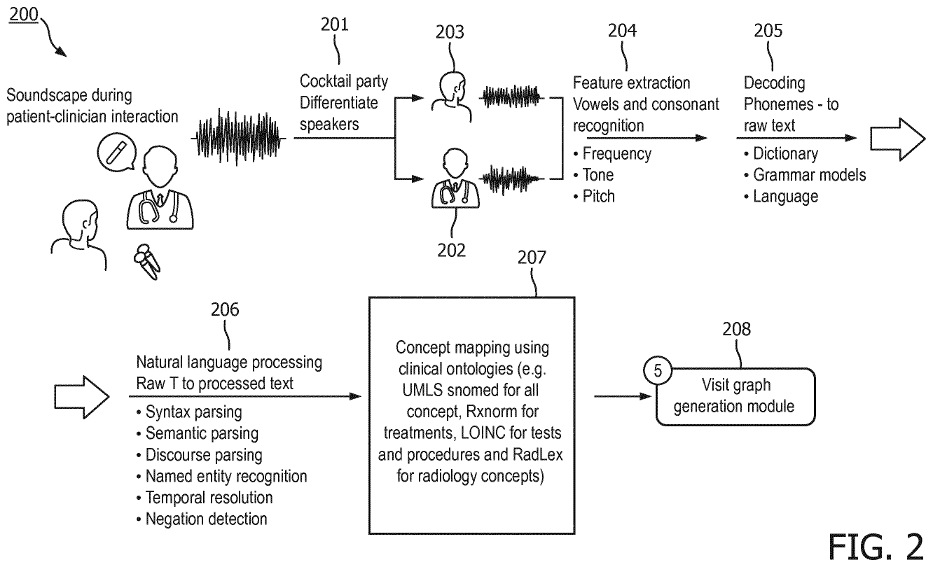

FIG. 2 illustrates a schema 200 for medical data information recorded from multiple data streams of patient-medical provider interaction to relevant information and events during visit of the current embodiment.

During information retrieval from the medical provider-patient interaction, the first step is automatic scribing that requires differentiation of the speaker. This may be achieved by using the cocktail party problem algorithm 201, which can be written as the following single line of code:

[W, s, v]=svd((repmat(sum(x.*x,1),size(x,1),1).*x)*x′);

The cocktail party problem algorithm 201 requires two microphones to differentiate the signals based on the spatial location of the speakers. The cocktail party algorithm 201 differentiates the two signals for the medical provider 202 and the patient 203.

Once the sound signals are isolated for the medical provider 202 and the patient 203, using cocktail party algorithm 201, deep neural networks may be used to perform recognition 204 and translation 205 of spoken information into raw text by using a model trained from a large amount of spoken language data.

During the second step, recognition 204, vowels and constants may be recognized using frequency, tone and pitch of the voice of the medical provider 202 and the patient 203.

During the third step, translation 205, phonemes are decoded to raw text using dictionaries, grammar models and language.

During the fourth step, a Natural Language Processing (“NLP”) 206 based classifier processes the raw text from the translation 205 into processed text by using syntax parsing, semantic parsing, discourse parsing, named entity recognition, temporal resolution and negation detection.

For example, the NLP 206 may use an introduction as a medical provider (e.g., “Hello, I am Dr. Smith”), use of complex clinical terms, and beginning with a question (e.g. “How are you feeling today?”) to identify which text belongs to a patient, medical provider, or care giver.

Further, a speech act classifier may be used to analyze the conversation structure at various levels (e.g., locutionary, illocutionary, and perlocutionary) that may enable understanding of the clinical scenario discussed between the patient and the medical provider.

The fifth step, mapping using clinical ontologies 207, uses the processed text information from the NLP 206 and events from the conversation during the visit and extracting them based on clinical ontologies and the event graph is generated 208.

The atomic actions and CPT codes database 104 includes a database of all possible “atomic actions” and CPT codes for a specific controlled environment. Network monitoring module 101 and the atomic action video recognition module 102 use the atomic actions and CPT codes database 104 to update their list of actions which could detect. The atomic actions and CPT codes database 104 may be used for storing results from the network monitoring module 101 or the atomic action video recognition module 102 which may provide continuous improvement of the system 100.

The visit graph generation module 105 may receive input from the network monitoring module 101, the atomic action video recognition module 102, the patient-provider conversation recognition module 103 and the atomic actions and CPT codes database 104 and creates and outputs an “event graph” for each visit.

An event graph is a set of atomic actions joined by relations including temporal ordering (e.g., action A occurred before action B), task hierarchy (e.g., action A and action B are steps in the same procedure), or contingency and causality (e.g., action B was necessary because of the results of action A).

The visit graph generation module 105 matches the detected medical equipment's 109 activities and atomic actions detected from the video with all possible atomic actions from the atomic actions and CPT codes database 104 and may use them for constructing the event graph. The visit graph generation module 105 may use a template event graph of a predefined or previous, positively rated, visits in construction of the event graph, for example, reducing uncertainty in action recognition by predicting expected next actions from the template event graphs.

The medical document generator 106 receives the event graph from the visit graph generation module 105 and translates the event graph to a draft of a medical note for a visit as a summarized, but comprehensive, for example, H&P. The medical document generator 106 may then match concepts or events based on a similarity score to a database of categorized concepts from previously acquired H&Ps.

The concepts may be mapped using clinical ontologies, for example, from the Unified Medical Language System (“UMLS”) including Systemized Nomenclature of Medicine (“SNOMED”) for all concepts, RxNorm for all treatments, Logical Observation Identifiers Names and Codes (“LOINC”) for tests and procedures, and Radiology Lexicon (“RadLex”) for radiology concepts.

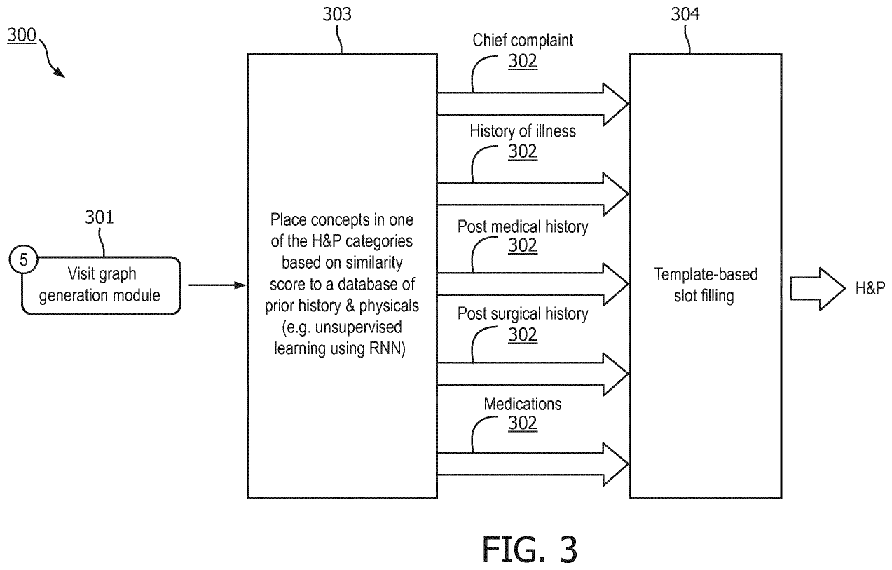

FIG. 3 illustrates a schema 300 of a H&P generator from a visit graph of the current embodiment.

The medical document generator receives the event graph from the graph generation module 301 and matches concepts or events based on a similarity score to a database of H&P categorized concepts 302 from previously acquired H&Ps. For example, H&P categories may include chief complaint, history of illness, post medical history, post surgical history and medications.

For example, matching the concepts or events based on a similarity score may be through recurrent neural networks (“RNN”) which may learn features of concepts and then use a simple logistic regression to place those features in the correct category 303. This may be performed by using a template based approach with a slot filling task 304.

In addition, the medical document generator module may use Centers for Medicare and Medicaid Service (“CMS”) guidelines to suggest an appropriate CPT codes for the medical visit.

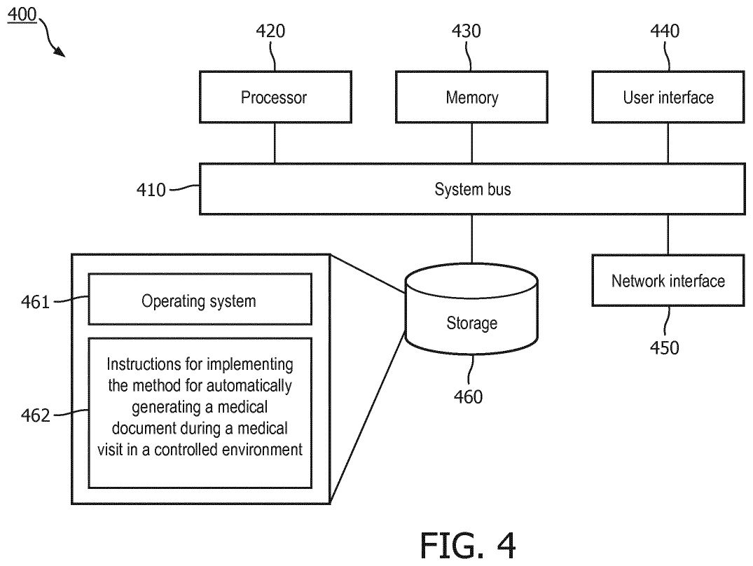

FIG. 4 illustrates an exemplary hardware diagram 400 for implementing a method for hybrid trust management for health records audit. As shown, the device 400 includes a processor 420, memory 430, user interface 440, network interface 450, and storage 460 interconnected via one or more system buses 410. It will be understood that FIG. 1 constitutes, in some respects, an abstraction and that the actual organization of the components of the device 400 may be more complex than illustrated.

The processor 420 may be any hardware device capable of executing instructions stored in memory 430 or storage 460 or otherwise processing data. As such, the processor may include a microprocessor, field programmable gate array (FPGA), application-specific integrated circuit (ASIC), or other similar devices.

The memory 430 may include various memories such as, for example L1, L2, or L3 cache or system memory. As such, the memory 430 may include static random access memory (SRAM), dynamic RAM (DRAM), flash memory, read only memory (ROM), or other similar memory devices.

The user interface 440 may include one or more devices for enabling communication with a user such as an administrator. For example, the user interface 440 may include a display, a mouse, and a keyboard for receiving user commands. In some embodiments, the user interface 440 may include a command line interface or graphical user interface that may be presented to a remote terminal via the network interface 450.

The network interface 450 may include one or more devices for enabling communication with other hardware devices. For example, the network interface 450 may include a network interface card (NIC) configured to communicate according to the Ethernet protocol. Additionally, the network interface 450 may implement a TCP/IP stack for communication according to the TCP/IP protocols. Various alternative or additional hardware or configurations for the network interface 450 will be apparent.

The storage 460 may include one or more machine-readable storage media such as read-only memory (ROM), random-access memory (RAM), magnetic disk storage media, optical storage media, flash-memory devices, or similar storage media. In various embodiments, the storage 460 may store instructions for execution by the processor 420 or data upon with the processor 420 may operate. For example, the storage 460 may store a base operating system 461 for controlling various basic operations of the hardware 400 and instructions for implementing method for automatically generating a medical document during a medical visit in a controlled environment 462.

It will be apparent that various information described as stored in the storage 460 may be additionally or alternatively stored in the memory 430. In this respect, the memory 430 may also be considered to constitute a “storage device” and the storage 460 may be considered a “memory.” Various other arrangements will be apparent. Further, the memory 430 and storage 460 may both be considered “non-transitory machine-readable media.” As used herein, the term “non-transitory” will be understood to exclude transitory signals but to include all forms of storage, including both volatile and non-volatile memories.

While the host device 400 is shown as including one of each described component, the various components may be duplicated in various embodiments. For example, the processor 420 may include multiple microprocessors that are configured to independently execute the methods described herein or are configured to perform steps or subroutines of the methods described herein such that the multiple processors cooperate to achieve the functionality described herein. Further, where the device 400 is implemented in a cloud computing system, the various hardware components may belong to separate physical systems. For example, the processor 420 may include a first processor in a first server and a second processor in a second server.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A method for automatically generating a medical document during a medical visit in a controlled environment, the method comprising the steps of:
monitoring, by a network monitoring module, a network to capture use of medical equipment connected to the network;
detecting, by an atomic action video recognition module, predefined atomic actions in the controlled environment;
extracting, by a patient-medical provider conversation recognition module, clinical information from a conversation between a patient and a medical provider;
matching, by a visit graph generation module, the use of medical equipment and the predefined atomic actions to an atomic actions and CPT codes database of known uses of medical equipment and predefined atomic actions;
generating, by the visit graph generation module, an event graph based on the use of medical equipment, the predefined atomic actions and the extracted clinical information; and
translating, by a medical document generator, the event graph into a medical document.

2. The method of claim 1, the method further comprising the steps of:
transmitting, by a communication interface, the medical document to the medical provider for review.

3. The method of claim 1, wherein the medical document generator improves the translation of the event graph into the medical document by capturing the changes made by the medical provider.

4. The method of claim 1, wherein the network monitoring module monitors the use of medical equipment by monitoring transactions in an electronic medical record (“EMR”) to extract the use of medical equipment.

5. The method of claim 1, wherein the patient-medical provider conversation recognition module extracts clinical information from the conversation by using an algorithm to differentiate the patient and the medical provider, extracting features from the conversation, decoding phonemes from the conversation to raw text, using natural language processing (“NLP”) to convert the raw text to processed text and mapping the processed text into a plurality of concepts using clinical ontologies.

6. The method of claim 1, wherein the event graph includes connecting the atomic actions detected by the atomic actions video recognition module to other atomic actions using temporal ordering.

7. The method of claim 1, wherein the visit graph generation module uses a template event graph to generate the event graph.

8. The method of claim 1, wherein the medical document generator categorized the concepts from the event graph into categories based on a similarity score for each of the plurality of concepts using recurrent neural networks.

9. The method of claim 1, wherein the medical document generator uses template based slot filling to generate the medical document from the categories.

10. The method of claim 1, wherein the medical document generator proposes a current procedural terminology code for the medical visit based on the medical document.

11. A system for automatically generating a medical document during a medical visit in a controlled environment, the system comprising:
a network monitoring module configured to monitor a network to capture use of medical equipment connected to the network;
an atomic action video recognition module configured to detect predefined atomic actions in the controlled environment;
a patient-medical provider conversation recognition module configured to extract clinical information from a conversation between a patient and a medical provider;
a visit graph generation module configured to match the use of medical equipment and the predefined atomic actions to an atomic actions and CPT codes database of known uses of medical equipment and predefined atomic actions;
the visit graph generation module configured to generate an event graph based on the use of medical equipment, the predefined atomic actions and the extracted clinical information; and
a medical document generator configured to translate the event graph into a medical document.

12. The system of claim 11, the system further comprising:
a communication interface configured to transmit the medical document to the medical provider for review.

13. The system of claim 11, wherein the medical document generator improves the translation of the event graph into the medical document by capturing the changes made by the medical provider.

14. The system of claim 11, wherein the network monitoring module monitors the use of medical equipment by monitoring transactions in an electronic medical record (“EMR”) to extract the use of medical equipment.

15. The system of claim 11, wherein the patient-medical provider conversation recognition module extracts clinical information from the conversation by using an algorithm to differentiate the patient and the medical provider, extracting features from the conversation, decoding phonemes from the conversation to raw text, using natural language processing (“NLP”) to convert the raw text to processed text and mapping the processed text into a plurality of concepts using clinical ontologies.

16. The system of claim 11, wherein the event graph includes connecting the atomic actions detected by the atomic actions video recognition module to other atomic actions using temporal ordering.

17. The system of claim 11, wherein the visit graph generation module uses a template event graph to generate the event graph.

18. The system of claim 11, wherein the medical document generator categorized the concepts from the event graph into categories based on a similarity score for each of the plurality of concepts using recurrent neural networks.

19. The system of claim 11, wherein the medical document generator uses template based slot filling to generate the medical document from the categories.

20. The system of claim 11, wherein the medical document generator proposes a current procedural terminology code for the medical visit based on the medical document.
